(19) United States
(12) Patent Application Publication
(10) Pub. No.: US 2010/0204518 A1
(43) Pub. Date: Aug. 12, 2010

(54) SINTERING RESISTANT CATALYST FOR USE IN HYDROGENATION AND DEHYDROGENATION REACTIONS AND METHODS FOR PRODUCING THE SAME

(75) Inventors: Aurel Wolf, Wulfrath (DE); Leslaw Mleczko, Dormagen (DE); Jens Assmann, Hilden (DE); Frank Rauscher, Koln (DE)

Correspondence Address: GERSTENZANG, WILLIAM C.;NORRIS MCLAUGHLIN & MARCUS, PA, 875 THIRD AVE, 8TH FLOOR, NEW YORK, NY, 10022, US

(73) Assignee: BAYER TECHNOLOGY SERVICES GMBH, LEVERKUSEN (DE)

(21) Appl. No.: 12/677,157

(22) PCT Filed: Sep. 9, 2008
(86) PCT No.: PCT/EP08/07954

§ 371 Date: Mar. 9, 2010

(30) Foreign Application Priority Data
Oct. 4, 2007 (DE) 10 2007 047 434.4

(51) Int. Cl. C07C 209/00; B01J 23/44; B01J 21/08

(52) U.S. Cl. 564/450; 502/339; 502/242; 977/773

(57) ABSTRACT

The invention relates to a novel thermostable palladium catalyst, a method for producing the same and the use thereof in hydrogenation reactions, especially hydrogenations of nitro compounds.

The invention relates to a novel thermally stable palladium catalyst, a process for producing it and its use in hydrogenations, especially hydrogenations of nitro compounds.

Supported heterogeneous noble metal catalysts play a critical role in many fields of chemical production, in particular in the field of hydrogenations and dehydrogenations. To obtain a high activity, the catalytically active components are applied in a finely divided fashion in the form of very small metal clusters (a few nm in size) to a support. This method gives a large specific surface area of metal which leads to a high catalytic activity. A disadvantage is that sintering, i.e. a growing together of the metal particles, very frequently takes place at the reaction temperature of the catalytic processes because of the mobility at relatively high temperatures (Ertl et. al., Handbook of Heterogenous Catalysis, 1997, vol. 3, 1276-1278). This induces a decrease in the catalytically active metal surface area, i.e. the catalytic activity decreases.

A slowing of the sintering process has been achieved in individual cases by optimization of the interaction between support and metal clusters or by addition of promoters.

However, since these known solutions can prevent sintering only to a certain degree, there is still a need for new types of thermally stable catalysts which, due to their structure, prevent sintering.

It was therefore an object of the invention to develop thermally stable palladium catalysts which due to their specific structure can completely prevent sintering. The activity of the catalyst should in this way be maintained over a very long period of time.

The object is surprisingly achieved by the inventive catalyst described below which is made up of nanoparticulate palladium and a porous zirconium oxide shell.

A similar structural principle is already known for gold catalysts (Arnal et. al., Angew. Chem., 2006, 118, 8404-8407) which are used in CO oxidation. However, palladium-based catalysts which are based on this or a similar principle are not known at present. This is probably attributable to the high tendency of gold to form nanoparticles compared to other metals, which significantly simplifies production of such catalysts.

The invention provides a catalyst based on at least one palladium nanoparticle having a gas- and liquid-permeable shell comprising zirconium oxide for use in hydrogenations and dehydrogenations.

The palladium nanoparticle has an average of the particle size distribution (d₅₀) which is preferably in the range 0.1-100 nm and particularly preferably 0.3-70 nm and very particularly preferably in the range 0.5-30 nm.

The internal diameter of the shell comprising zirconium oxide is preferably 10-1000 nm, very preferably 15-500 nm and very preferably 20-300 nm.

The layer thickness of the shell comprising zirconium oxide is usually in the range from 10 to 100 nm, preferably from 15 to 80 nm, particularly preferably 15-40 nm.

In a typical embodiment, the catalyst of the invention has many palladium nanoparticles having a gas- and liquid-permeable shell comprising zirconium oxide.

The invention further provides a process for producing a catalyst, which comprises the steps:
a.) production of palladium nanoparticles
b.) envelopment of the palladium nanoparticles produced with SiO₂
c.) application of a porous zirconium oxide layer to the Pd/SiO₂ spheres
d.) washing-out of the SiO₂ layer by means of a base

The catalyst is produced using palladium nanoparticles which are produced by reduction of a palladium-containing precursor in the liquid phase.

The production of the palladium nanoparticles in step a) is particularly preferably carried out using palladium salts which are soluble in alcohols, for example PdCl₂, H₂PdCl₄, Pd(NO₃)₂, palladium(II) trifluoroacetate, bis(acetonitrile)palladium(II) chloride and palladium(II) hexafluoroacetylacetonate, as palladium-containing precursor.

The reduction of the palladium-containing precursor can be carried out chemically and/or electrochemically. Preference is given to using reducing compounds having “active hydrogen”, e.g. hydrogen, methanol, ethanol, propanol and longer-chain alcohols, ethanediol, glycol, 1,3-propanediol, glycerol and polyols. Particular preference is given to using methanol, ethanol, propanol and polyols for reducing the palladium-containing precursor.

The particle size and particle size distribution can be influenced via the ratio of palladium-containing precursor and reducing agent.

The reduction of the palladium-containing precursor is usually carried out at temperatures of 0-250° C., preferably 10-200° C. and particularly preferably temperatures of 15-150° C.

The reduction of the palladium-containing precursor can take place either in the absence or presence of a surface-active stabilizer (also referred to as surfactants). However, the synthesis of the palladium nanoparticles preferably takes place using stabilizers which prevent agglomeration of the palladium nanoparticles and allow controlled setting of the particle size and morphology of the nanoparticles. Preference is given to using colloidal stabilizers such as polyvinylpyrrolidone (PVP), alcohol polyethylene glycol ethers (e.g. Marlipal®), polyacrylates, polyols, long-chain n-alkyl acids, long-chain n-alkyl acid esters, long-chain n-alkyl alcohols and ionic surfactants (e.g. AOT, CTAB) for this purpose. The mixing of palladium-containing precursors and stabilizer with the reducing compound can be carried out either in the semibatch mode or continuously in the liquid phase using suitable thermostated reactors (e.g. stirred tank reactor, flow reactor with static mixing internals, microreactors). In addition, the starting materials mentioned for producing the palladium nanoparticles can also be dissolved in the droplet volumes of liquid-liquid emulsions (e.g. miniemulsions or microemulsions) and then be reacted by mixing the two emulsion solutions.

The palladium colloids obtained by one of the methods described preferably have a very narrow distribution of the particle size, with the average of the particle size distribution (d₅₀) preferably being in the range 0.1-100 nm and particularly preferably 0.3-70 nm and very particularly preferably in the range 0.5-30 nm. The use of the abovementioned stabilizers enables the palladium nanoparticles to be redispersed in a suitable solvent after they have been separated off from the reaction solution (e.g. by ultrafiltration or by centrifugation). Preference is here given to using a solvent which is suitable for application of an SiO₂ layer, e.g. water, methanol, ethanol and further alcohols.

In step b), the palladium nanoparticles produced in step a) are, after having been separated off by centrifugation, sedimentation, etc., enveloped in a silicate shell. The envelopment with SiO₂ can be effected by hydrolysis or precipitating-on of hydrolyzable Si precursor. As hydrolyzable Si precursor, preference is given to tetramethyl orthosilicate, tetraethyl orthosilicate, tetrapropyl orthosilicate or similar hydrolyzable Si compounds.

The hydrolysis can preferably be carried out by means of a hydrolysis liquid containing ammonia solution, methanol, ethanol, propanol, isopropanol, butanol, 1,3-propanediol, glycerol, etc., or mixtures thereof.

The hydrolysis can, in particular, be carried out from room temperature (20° C.) to the boiling point of the hydrolysis liquid. The hydrolysis is very particularly preferably carried out at room temperature.

The diameter of the Pd-SiO₂ particles obtained in step b) is preferably 10-1000 nm and very preferably 15-500 nm and very preferably 20-300 nm. For further processing, the Pd-SiO₂ particles are preferably purified by cycles of separation from the liquid phase by, for example, sedimentation, centrifugation or evaporation and washing with washing liquids.

In step c), the preferably spherical Pd-SiO₂ nanoparticles produced in step b) are completely enveloped by a gas- and liquid-permeable shell comprising zirconium oxide. The envelopment with ZrO₂ can be effected by hydrolysis or precipitating-on of a hydrolyzable Zr precursor. Preferred hydrolyzable Zr precursors are zirconium alkoxides such as zirconium methoxide, zirconium ethoxide, zirconium n-propoxide, zirconium n-butoxide, or else zirconium halides such as ZrCl₄, ZrBr₄, ZrI₄ or similar hydrolyzable Zr compounds.

The hydrolysis can preferably be carried out by means of compounds having active hydrogen atoms, e.g. water, methanol, ethanol, propanol, glycerol, etc. The hydrolysis is very preferably carried out in the presence of colloid stabilizers such as alcohol polyethylene glycol ethers (e.g. Marlipal®), PVP, polyacrylates, polyols, long-chain n-alkyl acids, long-chain n-alkyl acid esters, long-chain n-alkyl alcohols. The hydrolysis can be carried out at temperatures of 0-200° C. Particular preference is given to using temperatures of 10-100° C. The thickness of the zirconium oxide layer can be set via the amount of hydrolyzable Zr precursor used.

After hydrolysis of the Zr precursor, aging over a period of from one hour to five days is preferably carried out. The particles are subsequently separated from the liquid by customary industrial methods (centrifugation, sedimentation, filtration, etc.) and dried in an oven and subsequently calcined. Drying can be carried out separately from the calcination in two separate steps or by increasing the temperature stepwise from room temperature to calcination temperature. Drying is preferably carried out in the temperature range 100-250° C., while calcination can be preferably be carried out at temperatures of 250-900° C.

In step d), the SiO₂ shell is removed from the essentially spherical Pd-SiO₂-ZrO₂ having a shell structure which is produced in step c). The removal of the SiO₂ is preferably carried out by dissolving the SiO₂ by means of a basic solution. As basic component of the solution, it is possible to use all alkali metal and alkaline earth metal hydroxides such as NaOH, KOH, LiOH, Mg(OH)₂, Ca(OH)₂, etc. The solution can be aqueous or alcoholic (MeOH, EtOH, PrOH, i-PrOH, etc.). The dissolution of the SiO₂ core usually occurs at temperatures of 0-250° C. and preferably at temperatures of 10-100° C. The alkaline solution is allowed to react until the SiO₂ core has been completely dissolved. This usually requires action of the alkaline solution over a period of 2-24 hours. Preference is also given to carrying out step d) a plurality of times using fresh alkaline solution.

Subsequent to step d), the Pd-ZrO₂ nanoparticles obtained are usually separated off and dried. The separation is preferably effected by centrifugation, filtration or sedimentation. Drying is preferably carried out in a stream of air at temperatures of 100-250° C. As an alternative, drying can also be carried out under protective gas or hydrogen.

In a further preferred embodiment of the process, the catalyst which is initially present in powder form is processed to produce shaped bodies. Preference is given to producing shaped bodies in the form of spheres, rings, stars (trilobes or tetralobes), pellets, cylinders or wagon wheels. The dimensions are preferably 0.2-10 mm, very preferably 0.5-7 mm. Processing is carried out by known methods such as pressing, spray drying and extrusion, in particular in the presence of a binder. A further preferred alternative is application of the catalyst of the invention as washcoat to structured catalysts (monoliths).

The Pd-SiO₂ nanoparticles according to the invention are suitable for use as thermally stable catalysts. Due to the ZrO₂ barrier, sintering of the Pd nanoparticles is not possible, meaning that the operating life and the cycle time under process conditions can be significantly increased compared to conventional catalysts. The increase in production time (elimination of catalyst regeneration) or lengthening of the production cycles enables the production costs of the hydrogenations or dehydrogenations to be significantly reduced.

The invention further provides for the use of the catalyst of the invention in hydrogenations of nitro compounds such as nitrobenzene, of alkenes such as ethylene, propylene, butene, butadiene, styrene, α-methylstyrene, in ring hydrogenations such as benzene to cyclohexane, naphthalene to decalin, in hydrogenations of nitrile compounds to amines, etc. The hydrogenations can be carried out at temperatures of 100-800° C. and very preferably at temperatures of 150-700° C. in the gas phase. Preference is given to using hydrogen as hydrogenation reagent. Limiting factors here are the stability of the compounds to be hydrogenated or of the products and also the vapor pressures of the reaction components or the pressure resistance of the reaction apparatuses. Hydrogenations are usually carried out at pressures of 1-200 bar.

The invention further provides for the use of the catalyst of the invention in transfer hydrogenations of nitro compounds such as nitrobenzene, dinitrobenzene, dinitrotoluene, nitrotoluene, nitrochlorobenzenes, nitronaphthalene, dinitronaphthalene, etc. The hydrogenations can, depending on the process (liquid phase or gas phase), be carried out at temperatures of 100-600° C.

The invention further provides for the use of the catalyst of the invention in hydrogenations such as propane to propylene, ethane to ethylene, butane to butene and butadiene and ethylbenzene to styrene.

The invention additionally provides a hydrogenation process for converting nitrobenzene into aniline by means of hydrogen in the gas phase in the presence of a catalyst, characterized in that a catalyst according to the invention is used.

The catalytic hydrogenations or dehydrogenations can preferably be carried out adiabatically or isothermally or approximately isothermally, batchwise, but preferably continuously as moving-bed or fixed-bed processes, preferably over heterogeneous catalysts at a reactor temperature of from 100 to 800° C., preferably from 150 to 700° C., particularly preferably from 200 to 650° C., and a pressure of from 1 to 250 bar (10 000 to 250 000 hPa), preferably from 1 to 200 bar. Customary reaction apparatuses in which the catalytic hydrogenations or dehydrogenations are carried out are fixed-bed or fluidized-bed reactors. The catalytic hydrogenations or dehydrogenations can also preferably be carried out in a plurality of stages.

In the adiabatic, isothermal or approximately isothermal mode of operation, it is also possible to use a plurality of, i.e. from 2 to 10, preferably from 2 to 6, particularly preferably from 2 to 5, in particular 2 to 3, reactors connected in series with intermediate cooling or heating. In the case of hydrogenations, the hydrogen can either all be added together with the reactants upstream of the first reactor or its addition can be distributed over the various reactors. This arrangement of individual reactors in series can also be combined in one apparatus.

### EXAMPLES

### Example 1

### Production of Palladium Nanoparticles—Step a):

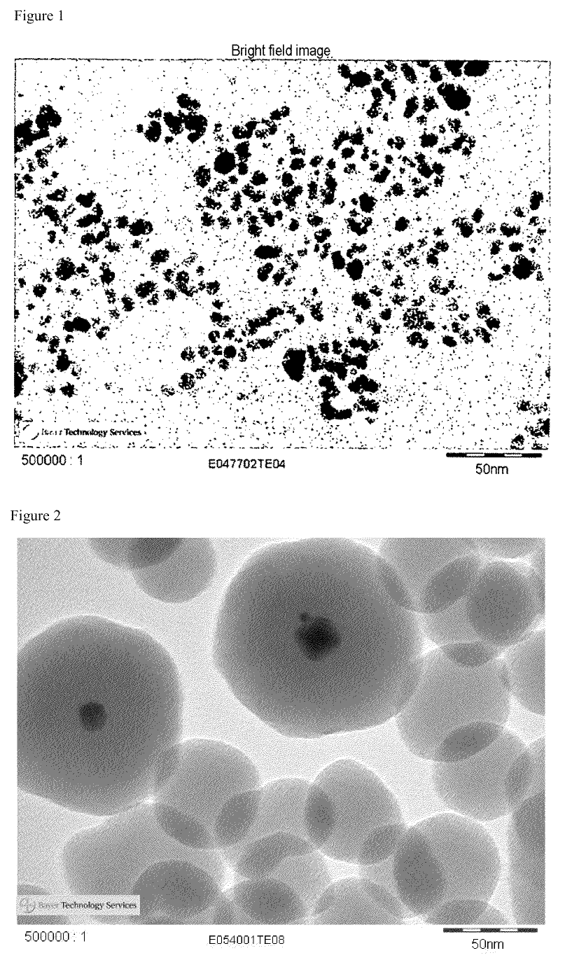

In a flask provided with magnetic stirrer, condenser and heating device, 106.4 mg (2.0 mmol) of PdCl₂ are mixed with 6 ml of HCl (0.2M) and 294 ml of distilled H₂O. This gives about 300 ml of a 2.0 mM H₂PdCl₄ solution. 15 ml (30 μmol of Pd) of the 2.0 mM H₂PdCl₄ solution are mixed with 31.5 ml of H₂O and 3.5 ml of methanol in a 100 ml flask. In addition, 300 μmol (33.25 mg) of PVP 40 (Sigma-Aldrich) are added and the total mixture is heated under reflux (temperature=80° C.) for 3 hours in an air atmosphere. The solution becomes brown immediately on heating. The cooled solution with the precipitated palladium nanoparticles is centrifuged at 10 000 rpm. The supernatant liquid is subsequently decanted off. The moist palladium particles can be used in this form for the further synthesis. FIG. 1 shows a transmission electron micrograph (instrument: Tecnai 20 LaB₆ cathode, camera: Tietz F114T 1×1, from FEI/Philips; method according to the manufacturer's instructions) of the palladium nanoparticles obtained. The average particle diameter is 8 nm.

Production of Pd-SiO₂ Nanoparticles—step b):

The palladium nanoparticles from step a) are redispersed in 3 ml of H₂O (ultrasonic bath: 10 min). Before commencement of the synthesis, the following solutions have to be prepared:
a. ethanol-NH₃ solution (total 10.5 ml): 0.5 ml of concentrated ammonia solution (28-30%) are mixed with 10 ml of ethanol
b. ethanol-TEOS solution (total 7.6 ml): 0.6 ml of tetraethyl orthosilicate are mixed with 7 ml of ethanol.

The aqueous palladium nanoparticle dispersion (3 ml) is stirred vigorously (5 min) The ethanol-NH₃ mixture is subsequently added Immediately thereafter, the ethanol-TEOS mixture is added very rapidly. The reaction mixture is stirred overnight at room temperature (20° C.). The Pd-SiO₂ nanoparticles are centrifuged (10 000 rpm; 25 min) and washed twice with water and once with absolute ethanol by, in each case, decanting off the supernatant liquid after centrifugation and redispersing the solid which remains (colloids) in the appropriate washing liquid by means of an ultrasonic bath (5 min) before recentrifuging. Finally, the Pd-SiO₂ nanoparticles are taken up in absolute ethanol (40 g) and redispersed by means of an ultrasonic bath (5 min US bath). The Pd-SiO₂ nanoparticles obtained in this way can be stored or used directly in the next step. FIG. 2 shows a transmission electron micrograph (instrument: Tecnai 20 LaB₆ cathode, camera: Tietz F114T 1×1K, from FEI/Philips; method according to the manufacturer's instructions) of the Pd-SiO₂ nanoparticles obtained in this way. The average diameter of the Pd-SiO₂ nanoparticles obtained is 120 nm.

Production of Pd-SiO₂—ZrO₂ nanoparticles—step c)

Before commencement of the synthesis, a Marlipal® O13/40 solution (ethoxylated isotridecanol; from Sasol) is produced by dissolving 0.43 g of Marlipal® in 11 g of H₂O. The Pd-SiO₂ nanoparticles obtained in step b) (30 μmol metal batch) are dispersed in 40 g of ethanol and transferred by means of absolute ethanol (25 g) into a 100 ml flask closed by means of a septum and subsequently heated to 30° C. 0.125 ml (125 μl) of the previously made up aqueous Marlipal® solution is added to the stirred dispersion of the Pd—SiO₂ nanoparticles which has been heated to 30° C. After 30 minutes, 0.45 ml of zirconium n-butoxide (80% by weight in butanol) is added. After stirring for 4 hours, the liquid phase of the dispersion is replaced by water. For this purpose, the dispersion is centrifuged (10 000 rpm; 15 min), the supernatant solution is decanted off and the solid is, after the supernatant liquid has been taken off, redispersed in 25 ml of water (ultrasonic bath: 5 min) This sequence of centrifugation and redispersion is carried out three times. The particles are subsequently aged at room temperature for 2 days. The sample is subsequently dried and calcined under an air atmosphere in a furnace. For this purpose, the temperature is increased stepwise from 100° C. to 900° C. over a total time of 7.5 h.

Production of Pd—ZrO₂ Nanoparticles—step d):

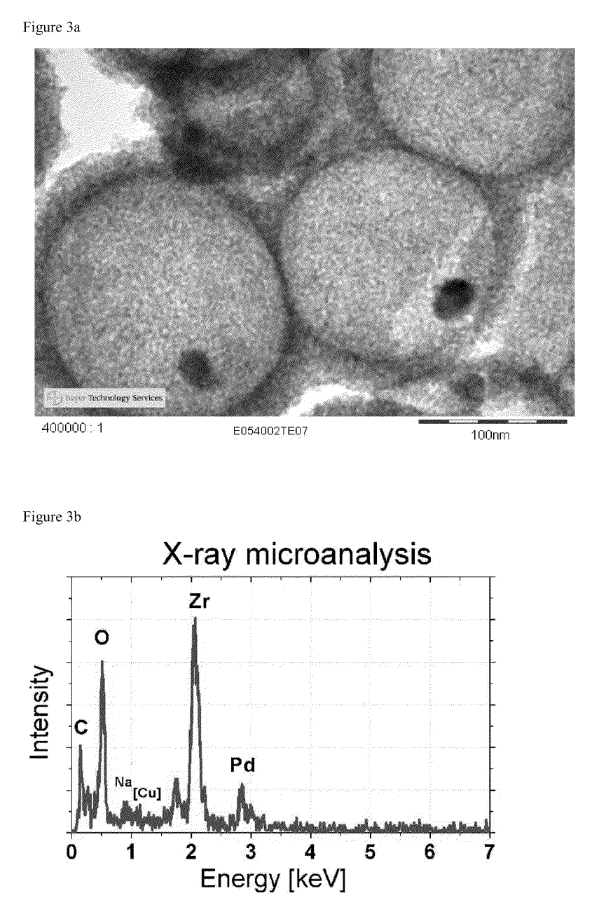

The Pd—SiO₂—ZrO₂ nanoparticles obtained in step c) (30 μmol metal batch) are stirred in 50 ml of 1 molar NaOH solution at room temperature for about 3 hours. The colloids are subsequently washed by centrifuging (10 000 rpm; 30 min), decanting off the supernatant liquid and taking up in 50 ml of 1-molar NaOH solution. The dispersion is stirred for 2 hours at 50° C. and subsequently overnight at room temperature. The particles are finally washed five times with water via a centrifugation/redispersion sequence. The Pd—ZrO₂ particles obtained in this way no longer have an SiO₂ core and in the porous shell have a sintering barrier. FIG. 3a shows a transmission electron micrograph (instrument: Tecnai 20 LaB₆ cathode, camera: Tietz F114T 1×1K, from FEI/Philips; method according to the manufacturer's instructions) and FIG. 3b shows the results of the XPS analysis (instrument: Phoenix, from EDAX/Ametek; method according to the manufacturer's instructions). The average diameter of the Pd—ZrO₂ particles is 130 nm. It can be seen from the XPS analysis that SiO₂ is no longer present in the nanoparticles.

1. A catalyst comprising at least one nanoparticulate palladium cluster and a gas- and liquid-permeable shell comprising zirconium oxide for use in hydrogenations and dehydrogenations.

2. The catalyst as claimed in claim 1, wherein the nanoparticulate palladium cluster has an average of the particle size distribution (d₅₀) in the range 0.1-100 nm and the shell comprising zirconium oxide has an internal diameter in the range 10-1000 nm.

3. The catalyst as claimed in claim 1, wherein the layer thickness of the shell comprising zirconium oxide is in the range from 10 to 100 nm.

4. A process for hydrogenating organic compounds with hydrogen in the gas phase in the presence of the catalyst of claim 1.

5. A method for hydrogenations or transfer hydrogenations of nitro compounds, or for dehydrogenation reactions, which comprises carrying out said hydrogenations, transfer hydrogenations or dehydrogenation reactions in the presence of the catalyst of claim 1.

6. The method of claim 5, wherein the hydrogenations of nitro compounds is carried out in the liquid or gas phase at temperatures of 100-600° C.

7. A process for converting nitrobenzene into aniline by hydrogen in the gas phase in the presence of the catalyst of claim 1.

8. A process for dehydrogenating organic compounds in the gas phase in the presence of a catalyst, wherein said catalyst is the catalyst claim 1.

9. A process for producing a catalyst, which comprises the steps of:
a.) producing palladium nanoparticles having an average of the particle size distribution (d₅₀) in the range 0.1-100 μm,
b.) enveloping the palladium nanoparticles produced with SiO₂ to produce Pd/SiO₂spheres,
c.) applying a layer of zirconium oxide to the Pd/SiO₂ spheres
d.) washing-out the SiO₂ layer with a base.

10. The process as claimed in claim 9, wherein the production of palladium nanoparticles in step a) is carried out by reduction of a palladium-containing precursor in the liquid phase in the presence of at least one colloidal stabilizer selected from the group consisting of polyvinylpyrrolidone, alcohol polyethylene glycol ethers, polyacrylates, polyols, long-chain n-alkyl acids, long-chain n-alkyl acid esters, long-chain n-alkyl alcohols and ionic surfactants.

11. The process as claimed in claim 9, wherein the application of a zirconium oxide layer in step c) is carried out by hydrolysis or precipitation of a hydrolyzable Zr precursor in the presence of at least one colloid stabilizer selected from the group consisting of alcohol polyethylene glycol ethers, polyvinylpyrrolidone, polyacrylates, polyols, long-chain n-alkyl acids, long-chain n-alkyl acid esters and long-chain n-alkyl alcohols.
